(19) United States
(12) Patent Application Publication
(10) Pub. No.: US 2024/0285480 A1
(43) Pub. Date: Aug. 29, 2024

(54) Product comprising a liquid composition and a solid composition comprising at least one cavity

(71) Applicant: CAPSUM, MARSEILLE (FR)

(72) Inventors: Sébastien BARDON, VEYRIER DU LAC (FR); Benjamin AUGAIS, ALLAUCH (FR)

(21) Appl. No.: 18/584,735

(22) Filed: Feb. 22, 2024

(30) Foreign Application Priority Data
Feb. 23, 2023 (FR) 23 01653

(51) Int. Cl. A61K 8/02; A61Q 19/00

(52) U.S. Cl. CPC A61K 8/0233; A61Q 19/00; A61K 2800/87

(57) ABSTRACT

A product includes at least one first composition and at least one second composition: the first composition is a solid composition comprising at least one cavity such that the composition has porosity greater than or equal to 20% by volume relative to the total volume of the solid composition; the second composition is a liquid composition, preferably in the form of a cream, and advantageously having viscosity higher than 5000 cps, and wherein all or part of the second composition is housed in all or part of the cavities of the first composition.

The invention relates to a product comprising at least one liquid composition and at least one solid composition having at least one cavity.

The fields of cosmetics, pharmaceuticals, nutraceuticals, or foodstuffs do not escape from the demand for novelty by consumers. However, in these fields, innovations generally concern efficiency and only rarely concern gestural, sensorial and galenic aspects.

From this perspective, the Applicant has developed microfluidic production methods giving access to emulsions having a dispersed phase in the form of macroscopic drops which may be monodisperse. Said methods are particularly described in WO2012120043 and WO2019145424.

In addition, the Applicant has developed solid compositions provided with a high level of porosity, giving access to compositions that are less raw material-intensive whilst still having satisfactory handling properties. Said solid compositions are particularly described in the patent applications filed under N°s FR2203883, FR2203882, FR2203887, FR2203886 and FR2206462.

Nonetheless, meeting consumer demand for novelty remains a constant pursuit. The present invention sets out to meet this demand, in particular with regard to gestural, sensorial and galenic aspects.

The invention therefore concerns a product comprising at least one first composition and at least one second composition, wherein:
the first composition is a solid composition comprising at least one cavity such that the first composition has porosity greater than or equal to 20% by volume relative to the total volume of the first composition,
the second composition is a liquid composition liquid, preferably in the form of a cream and advantageously having viscosity higher than or equal to 5000 cps,
and wherein all or part of the second composition is housed in all or part of the cavities of the first composition.

As follows from the examples below, a product of the invention is advantageous in that it provides access to:
a novel, differentiating galenic form;
a novel, differentiating design; and
a product allowing the use, within one same product, of potentially incompatible raw materials.

Additionally, when used via topical route, a product of the invention is advantageous in that:
it has unique sensorial handling, translating as alternation between softness and glide with the second composition, and resistance with the first composition. It is even possible to talk of saccade-like dispensing which is particularly unprecedented and gives satisfaction to users; and
the architecture of the product allows improved dispensing of the second composition, in particular when it is difficult to extract.

With these advantages, better compliance is seen in users to heed the routine for use/administration, which mechanically allows optimization of the desired effects in particular when the product is intended for cosmetic or pharmaceutical use.

A product of the invention is also advantageous in that it allows storage of at least part of the second composition in all or part of the cavities of the first composition, thereby allowing compartmenting of the second composition. This packaging mode ensures improved preserving of the second composition, and even reducing of preserving agent content if any.

If the second composition is scarcely viscous i.e. having viscosity of less than 10000 mPa·s, and preferably having a low flow threshold value for example lower than 1 Pa, in particular lower than 0.5 Pa, even lower than 0.1 Pa, a product of the invention is further advantageous in that the second composition is able to rise via capillarity into the cavities of the first composition, facilitating dispensing thereof. This makes it possible to overcome the disadvantages usually encountered by users when the product is nearly exhausted and extraction of the composition from the bottom of the holder can prove to be tedious and scarcely practical.

Unless otherwise stated, it is considered that temperature is ambient temperature (for example T=25° C.±2° C.) and pressure is atmospheric pressure (760 mm of Hg, i.e. 1.013×10⁵ Pa or 1013 mbar).

In the invention, the pH of product is typically between 4.0 and 8.0, in particular between 5.0 and 7.0.

### DESCRIPTION OF THE FIGURES

FIG. 1 illustrates a product of the invention in which the cavities of the first composition are honeycomb-shaped and the second composition is a direct emulsion.

FIG. 2 illustrates two first compositions respectively having round or triangular cavities.

### Product

A product of the invention comprises at least one first solid composition comprising at least one cavity such that the first composition has porosity greater than or equal to 20% by volume relative to the total volume of the first composition, and a second liquid composition.

### First Solid Composition

In the meaning of the present invention, the first composition can indifferently be designated as «first solid composition» or «solid composition».

For obvious reasons, the viscosity/solidity of the first composition and the formulation thereof are adjusted to allow easy dispensing of the product of the invention.

The first composition is a solid composition comprising at least one cavity such that the first composition has porosity greater than or equal to 20%, preferably greater than or equal to 30%, better still greater than or equal to 40%, in particular greater than or equal to 50%, even greater than 60%, in particular greater than or equal to 70%, and more particularly greater than or equal to 80% by volume relative to the total volume of the first composition.

By «solid», in the meaning of the invention, it is meant to designate a first composition which, at ambient temperature and atmospheric pressure, is not likely to collapse under its own weight. Advantageously, it is meant to designate any composition having a compressive strength higher than or equal to 20 g, preferably higher than or equal to 50 g, and better still higher than or equal to 100 g, at ambient temperature (20-25° C.), after penetration by a cylindrical probe of revolution having a diameter of 0.8 cm into the matrix of the composition to a thickness of 1 mm at a rate of 0.5 mm/s, and removal of said probe from the matrix of the composition at a rate of 0.5 mm/s; the compressive strength being measured with an analyzer of “LFRA Texture Analyzer” type marketed by STEVENS/MECHTRIC.

A first composition is not in liquid form.

A first composition is not in the form of a paste. Nevertheless, at the time of manufacture thereof, in particular if manufacture has recourse to an extrusion process such as described below, the first composition may be in the form of a paste.

A first composition is not a powdery composition, and is therefore in non-particulate form. A first composition of the invention is therefore not in powder form, in particular in the form of free or compacted powder. Finally, a first composition is not a porous solid either derived from the agglomeration of powders.

A first solid composition of the invention is therefore a non-powdery composition, and is not therefore in tablet or capsule form based on compacted powder.

In a first embodiment, a first composition is not an expanded composition and is therefore not a foam. In a second embodiment, a first composition can be an expanded composition and can therefore be in the form of a foam.

Advantageously, a first composition is not composed of a network of fibres.

Preferably, a first composition of the invention does not comprise an alcohol.

Preferably, a first solid composition of the invention does not comprise polyvinyl alcohol (PVA or PVOH), a polyacrylate, a copolymer of acrylic acid and methacrylic acid, a polyvinylidene fluoride (PVDF), polymethyl methacrylate (PMMA), polyvinylpyrrolidone, polyalkylene oxide, polyacrylonitrile (PES or PAN), polymethacrylate, polyethylene terephthalate, polyethylene, polypropylene, nylon, hyaluronic acid, starch and derivatives thereof, pullulan, gelatine, cellulose (e.g. rayon), hydroxypropylmethylcellulose, methylcellulose, carboxymethylcellulose, cotton, or one of the mixtures thereof.

Preferably, a first composition of the invention does not comprise (or is not based on) a water-soluble solid substrate e.g. of hydrogel type, in particular starch-based and possibly unstructured, and/or poloxamer 407. However, this does not prevent a solid composition of the invention from comprising starch as described below.

Advantageously, a first composition is not in the form of a mask.

Preferably, a first solid composition of the invention is not coated with a water-soluble coating.

The solid nature of a first composition can be based on a phase mostly composed of, even fully composed of, a fatty phase comprising at least one lipophilic gelling agent (also called below «solid fatty phase»).

Also, the solid nature of the first composition can be based on a phase derived from a saponification reaction (or «soap paste compounding»), and therefore follows from the conversion of a fatty phase comprising at least one oil, preferably vegetable oil, under the action of sodium hydroxide and optionally heat (also called hereafter «soap phase»).

Additionally, the solid nature of the first composition can be based on a phase comprising at least one synthetic detergent (or surfactant) (also called hereafter «SYNDET phase»). As an illustration, it is CETYL ALCOHOL, all or part thereof, which contributes to imparting the solid nature to a SYNDET.

The solid nature of the first composition can therefore be based on (i) a solid fatty phase, (ii) a soap phase, and/or (iii) a SYNDET phase. In the remainder of the description, the «solid fatty phase», the «soap phase» and «SYNDET phase» can be commonly designated by the expression «solid phase».

Therefore, a first composition comprises at least one solid phase.

Preferably, a first solid composition is single-phase and therefore solely formed of the solid phase.

In one particular embodiment, a first composition may also comprise an aqueous phase dispersed in the solid phase, preferably in encapsulated form, for example in the form of matrix spheres (or pearls or beads) or of core/shell type (or capsules), for example in the form of capsules such as described in WO2010063937.

Preferably a first composition of the invention is a single-dose composition. This embodiment is particularly advantageous from a hygienic viewpoint in that it prevents repeated contact between the first solid composition and the surface to be treated.

Preferably, a first composition of the invention is a topical composition. A first composition of the invention is not an oral composition.

In one embodiment, the mass percentage of water in the dispersed aqueous phase can then be between 0.5 and 50%, preferably between 1 and 40%, in particular between 2.5 and 30%, and better still between 5 and 20% by weight relative to the total mass of the first composition.

By «porosity», in the meaning of the present invention, it is meant to designate all the voids of a solid material, these voids able to be filled with the second composition.

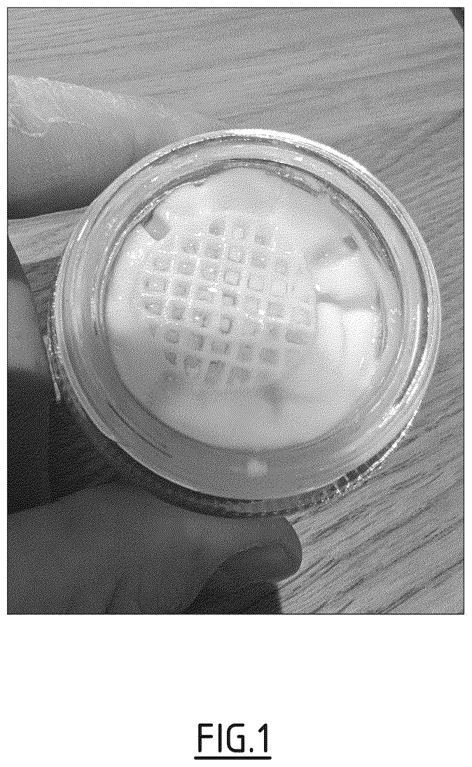

By «cavity», in the meaning of the present invention, it is meant to designate an empty space inside a solid body and which communicates directly with the outer medium, similar for example to the cavities illustrated in FIGS. 1 to 3 and 6 in the patent application filed under N° FR2203881. A cavity can therefore be indifferently designated by the terms «orifice», «pore» or «hole». Advantageously, the cavity/cavities of the first composition are through cavities i.e. the cavity designates an empty space inside a solid body and which communicates directly with the outer medium either side of the solid body.

Therefore, the volume of the cavity/cavities in a first composition represents at least 20%, in particular at least 30%, preferably at least 40%, more preferably at least 50%, even at least 60%, in particular at least 70%, and most particularly at least 80%, of the total volume of the first composition.

Advantageously, the density of a first composition is between 0.2 and 0.8, preferably between 0.3 and 0.7, in particular between 0.4 and 0.6.

A first composition can have a total volume of between 0.125 cm³ and 100 cm³, preferably between 0.250 cm³ and 75 cm³, in particular between 0.5 cm³ and 50 cm³, better still between 1 cm³ and 25 cm³, and more particularly between 2.5 cm³ and 15 cm³.

A first composition can have a weight of between 0.025 g and 200 g, preferably between 0.20 g and 150 g, in particular between 0.5 g and 100 g, better still between 1 g and 75 g, and more particularly between 2.5 g and 50 g, even between 5 g and 25 g («g» corresponding to the unit «gram»).

In one preferred embodiment, a first composition has a ratio between the volume of the cavities and the total volume of the first composition of between 0.05 and 0.9, preferably between 0.1 and 0.8, in particular between 0.2 and 0.9, preferably between 0.2 and 0.8, better still between 0.2 and 0.7, even between 0.2 and 0.6.

By «total volume», it is meant to designate the volume resulting from the sum of the volume of the solid and the volume of the cavities.

In the present invention, the cavities can be termed macroporous i.e pores having a minimum width or diameter greater than or equal to 0.5 mm, in particular greater than or equal to 0.75 mm, preferably greater than or equal to 1 mm, even between 0.5 mm and 2 cm, more particularly between 1 mm and 2 cm, preferably between 2 mm and 1 cm, in particular between 3 mm and 75 mm, better still between 4 mm and 50 mm, and more particularly between 5 mm et 25 mm. In particular, the pores have a minimum width, or diameter, of between 0.5 mm and 5 mm, preferably between 1 mm and 3 mm.

In this respect, the Applicant indicates that, for equivalent porosity, the manufacture of a stable solid composition is:
easy in the presence of cavities of small size, for example those contained in solid substrates in which porosity follows from the insertion of a gas during the manufacturing process, similar to the description in U.S. Pat. No. 8,349,341, but
much more difficult in the presence of macroporosities such as those considered in the present invention.

Advantageously, the size and/or volume of the cavities can be adjusted to store and/or dispense and/or administer the correct amount of second composition required to obtain a product of the invention having satisfactory/expected properties, for example in terms of viscosity, hydration, comfortable application and/or transformative effect.

A first solid composition can be of any shape, for example spherical, ellipsoid, tetrahedral or polygonal, in particular in the form of a parallelogram, preferably a rectangular parallelogram.

Preferably, to ensure good handling of a kit such as described below, a first composition has:
a width, or diameter, of between 1 cm and 10 cm, preferably between 2 cm and 8 cm, in particular between 3 cm and 6 cm, and better still between 4 cm and 5 cm; and/or
a height (or thickness) of between 0.5 cm and 5 cm, preferably between 1 cm and 4 cm, and in particular between 2 cm and 3 cm.

In one particular embodiment, a first composition may comprise additional patterns differing from the cavities, said patterns possibly being obtained by embossing for example.

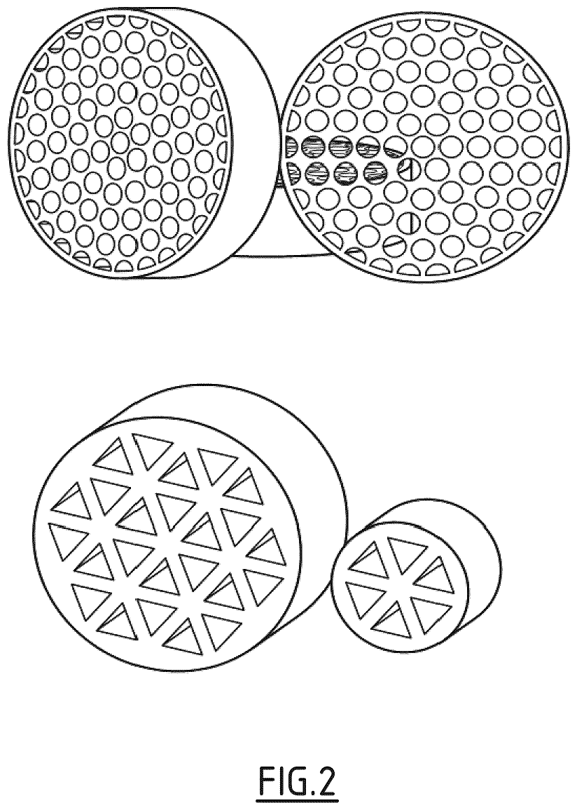

The cavity/cavities of the first composition can be of any shape for example, round, oblong, ovoid, triangular, polygonal in particular hexagonal, honeycomb or tessellated (or partitioned), for example of Voronoi diagram type, preferably of honeycomb shape such as illustrated in FIG. 2 or in FIGS. 3 and 6 of the patent application filed under n° FR2203881.

The cavity/cavities of a first composition can also represent letters or the logo of a trademark or company.

A first composition comprises one or more cavities, preferably several cavities, the same or different.

In a first variant, a first composition of the invention comprises at least one cavity in particular a through cavity.

In a second preferred variant, a first composition of the invention comprises several cavities, in particular through cavities, such as illustrated in FIG. 3 in the patent application filed under n° FR2203881.

Advantageously, the cavities are through cavities along an axis x-x′.

In one particular embodiment, a first composition may comprise at least two cavities connected together. Said embodiment is advantageous since it allows promoting of capillary phenomena described previously, which can allow an improvement in the aforementioned technical effects.

Advantageously, a first composition comprises several cavities having a structure of honeycomb shape such as illustrated in FIG. 1. This honeycomb shape is particularly advantageous in that it permits the manufacture of a first composition having a ratio of «volume of cavity(cavities)/total volume of first composition» that is highly favourable, in particular higher than or equal to 0.5, even higher than or equal to 0.75, without negatively affecting the handling and sturdiness of the first composition against mechanical stresses likely to be applied thereto and/or during storage. In other words, this honeycomb structure affords an excellent compromise between cavity volume and mechanical strength.

Advantageously, the cavity/cavities of a first solid composition of the invention comprise walls having a thickness of between 10 μm and 1500 μm, preferably between 20 μm and 1000 μm, in particular between 30 μm and 750 μm, more particularly between 50 μm and 500 μm, even between 100 μm and 400 μm. In particular, the cavity/cavities of a solid composition of the invention comprise walls having a thickness of between 250 μm and 500 μm.

Advantageously, a first solid composition comprises several cavities which adopt a honeycomb structure comprising walls having a thickness of between 10 μm and 600 μm, preferably between 20 μm and 500 μm, in particular between 30 μm and 400 μm, more particularly between 50 μm and 300 μm, even between 100 μm and 200 μm.

In a first variant, a first composition is anhydrous. By «anhydrous», in the meaning of the invention, it is meant to designate a composition which has a water content lower than 5% by weight, preferably lower than 1% by weight, and in particular lower than 0.5% by weight relative to the total weight of said composition.

Preferably, a first composition is free of surfactant.

Advantageously, a first solid composition of the invention does not comprise alkyl- and/or alkenyloligoglycosides, olefinsulfonates and/or dextrins, in particular maltodextrin, or nucleating agent, tale in particular.

Advantageously, a first solid composition of the invention does not comprise dextrins, in particular maltodextrin.

Advantageously, a first solid composition of the invention does not comprise water-insoluble particles, chosen in particular from among inorganic insoluble particles and more particularly chosen from among carbonates, sulfates, silicates, clays, aluminates, phosphates, sulfates, talc, and combinations thereof, and in particular such as described in GB2459093.

Advantageously, a first solid composition of the invention does not comprise polyethylene oxide, in particular such as described in U.S. Pat. No. 4,277,358.

### Method for Manufacturing the First Composition

A first composition can be obtained with any method known to persons skilled in the art and adapted to the manufacture of a solid composition, subject to the necessary adjustments to ensure the formation of cavities and therefore of meeting the required criterion of porosity.

Preferably the method for manufacturing a solid composition of the invention does not comprise an aeration/gas injecting step e.g. of air, possibly followed by a forming and drying step.

### Method N°1

In a first variant, a method for manufacturing a first composition comprises at least the steps of:

providing a fatty phase, a phase derived from a saponification reaction and/or from a phase comprising at least one synthetic detergent (or surfactant) (i.e. SYNDET phase), in liquid or semi-liquid form;
(ii) pouring the phase of step (i) into a mould and drying until a solid phase is obtained,
(iii) cutting the solid phase to the desired shape, and
(iv) forming cavities, after which a first composition is obtained.

In one particular embodiment, the method for manufacturing the first composition is based on a saponification method (or «soap paste compounding»).

Step (i) is advantageously conducted under heat.

Advantageously, steps (iii) and (iv) are simultaneous.

Advantageously, steps (ii), (iii) and (iv) are simultaneous, in which case the mould is of adapted shape to ensure forming of the cavity/cavities.

Step (iv) is advantageously performed using a punch.

Method N°1 is advantageous in that the offcuts obtained after step (iv) can be reused for subsequent manufacture. These offcuts can be reheated and fed into a subsequent process of method N°1 at step (i) and/or (ii).

Advantageously, in particular when the first composition is a soap and/or shampoo and therefore comprising a soap phase such as previously described, a method of the invention may also comprise, before step (ii), at least one wash step to remove remaining sodium hydroxide.

### Method N°2

In a second variant, a method for manufacturing a first composition is an extrusion method.

Said manufacturing method comprises at least the steps of:
providing a fatty phase, a phase derived from a saponification reaction and/or a phase comprising at least one synthetic detergent (or surfactant) (i.e. SYNDET phase), in liquid or semi-liquid form;
(b) passing the phase of step (a) into an extrusion device provided with a die (or extrusion grid) of adapted shape to form a solid bar, and
(c) cutting the solid bar obtained a step (b), after which a first composition is obtained.

Step (a) is advantageously conducted under heat.

For obvious reasons, step (b) is conducted at a temperature adapted so that the phase used has sufficient viscosity and is able to maintain the shape imparted by the die. Therefore, and if necessary, the phase injected into the extruding device is heated to change from a solid state to a paste state, thereby guaranteeing proper performing of this extrusion step.

For obvious reasons, the shape of the die of the extrusion device is also adapted to ensure the formation of the cavity/cavities.

At step (b), the extrusion device comprises a die provided with a shape adapted to provide the solid bar with the desired cavity/cavities and degree of porosity.

As an illustration, an extrusion method comprises a die such as the one illustrated in FIG. 4 of the patent application filed under n° FR2203882.

Advantageously, the die output temperature can be lower than ambient temperature, for example lower than or equal to 15° C., even lower than or equal to 10° C.

Step (c) can be performed with a knife or wire, for example a butter cutting wire.

Preferably, steps (b) and (c) are simultaneous.

Preferably, the method for producing a first composition is the extrusion method N°2 described above.

In one particular embodiment, applicable to methods N°1 and n°2 previously described, step (i)/(a) comprises providing a fluid phase which may comprise at least one solvent that is able to evaporate fully or partially at step (ii)/(b), even also at step (iii) and/or (iv)/(c).

As solvent, mention can be made for example of an apolar or scarcely polar inorganic solvent, for example cyclohexane, THF (tetrahydrofuran), and mixture thereof.

In the above methods N°1 and n°2, when the first composition comprises at least one fatty phase comprising at least one lipophilic gelling agent, step (i)/(a) may require heating the phase to a temperature higher than the highest melting point of the lipophilic gelling agents contained in the phase.

In one particular embodiment, extrusion method N°2 can be performed with a 3D printing technique.

### Second Liquid Composition

In the meaning of the present invention, the second composition can indifferently be designated as «second liquid composition» or «liquid composition».

In a first variant, the second composition is aqueous.

In a second variant, the second composition is anhydrous.

In a third variant, the second composition is an emulsion. An emulsion can be a direct emulsion (i.e. oil-in-water), invert emulsion (i.e. water-in-oil), or a multiple, in particular double emulsion (i.e. water-in-oil-in-water, oil-in-water-in-oil, or oil-in-oil-in-water).

In one particular embodiment, the second composition is an emulsion, preferably a direct emulsion, that is stable and highly concentrated in dispersed phase and in particular an emulsion of HIPE type (High Internal Phase Emulsion) or Apollonian emulsion.

In another particular embodiment, the emulsion is a macroemulsion i.e. in which the dispersed phase is in the form of drops visible to the naked eye, for example having a diameter of between 100 μm and 3000 μm, preferably between 250 μm and 1500 μm, kinetically stable and optionally monodisperse. The monodisperse nature and a measuring method are defined in FR3041511.

A second composition of the invention is not in solid form at ambient temperature and at ambient pressure i.e. it is able to flow under its own weight.

A second composition of the invention is in liquid form, preferably in gel form.

The viscosity of a second composition, or at least of the continuous phase thereof when in emulsion form, can vary widely which allows the obtaining of varied textures. Viscosity is measured at ambient temperature and ambient pressure according to the method described in WO2017046305.

For obvious reasons, the viscosity of the second composition is adjusted to allow easy dispensing/administering of the product of the invention.

In one embodiment, the second composition advantageously has viscosity from 5000 mPa·s to 500 000 mPa·s, preferably from 10 000 mPa·s to 300 000 mPa·s, better still from 25000 mPa·s to 100 000 mPa·s, and more particularly from 50 000 mPa·s to 75000 mPa·s, such as measured at 25° C. according to the above-described method.

The second composition is advantageously in the form of a gel or cream, and preferably in the form of a cream.

Preferably, a second composition of the invention is a topical composition. A second composition of the invention is not an oral composition.

Preferably, the second composition is able to reduce the solidity of the first composition. In particular, the second composition may advantageously comprise at least one raw material able to reduce the solidity of the first composition. This embodiment is advantageous in that it allows the solid texture of the first composition to evolve towards a softer texture. The term «transformative effect» can therefore be used.

Preferably, a second composition is free of surfactant. Against all expectations, the inventors have observed that the absence of surfactant in the second composition is able to enhance this transformative effect of the first composition, in particular the kinetics thereof.

A second composition can be prepared with any method known to persons skilled in the art.

A second composition, when in emulsion form, has the advantage of being able to be prepared using a simple «non-microfluidic» method, namely by simple emulsification in particular by means of an agitation device of Rayneri type or bladed agitator.

A second composition can also be prepared with a microfluidic method, in particular as described in applications WO2012/120043 or WO2019/145424. With this preparation method, the microfluid nozzle(s) used can have a configuration of T geometry in co-flow or flow-focusing.

In one particular embodiment, a product of the invention may comprise several first compositions, the same or different, and/or several second compositions, the same or different.

As an illustration of this particular embodiment:
in a first variant of embodiment, a product of the invention may comprise a first composition comprising a second composition A housed in the cavities of the first composition, and a second composition B preferably differing from second composition A, located outside the first composition;
in a second variant of embodiment, a product of the invention may comprise two first compositions C and D, preferably different, respectively comprising a second composition A and a second composition B, preferably the second compositions A and B being different, and even optionally a second composition E preferably differing from the second compositions A and B and located outside the first compositions C and D.

In this particular embodiment, each of the first and/or second composition(s) can particularly comprise a different biological/cosmetic active substance and preferably a different colouring agent, even the first compositions can comprise cavities of different shapes, thereby enabling the user to easily identify which product it is desired to apply and therefore which effect, in particular cosmetic effect, it is desired to obtain.

For a cosmetic product, this particular embodiment allows the affording of a product having different cosmetic effects, thereby allowing personalized use and in particular easy adaptation for users according to state of mind (mood), dress style and/or time of day.

A product of the invention may advantageously comprise a weight ratio of «first composition(s)/second composition(s)» of between 0.01 and 10, preferably between 0.1 and 5, better still between 0.5 and 1.

### Third Composition

In one particular embodiment, a product of the invention may further comprise at least one third composition, preferably solid at ambient temperature, dedicated to closing all or part of the cavities of the first composition, and preferably all or part of the upper portion of the cavities of the first composition, similar to films used by bees for example to close the alveoli full of honey.

By «upper portion of the cavities», it is meant to designate the outlet orifices of the cavities facing the orifice of the container comprising the product of the invention, and therefore generally the outlet orifices of the cavities in contact with ambient air. In other words, the third composition covers all or part of the first and second compositions.

Preferably, the third composition is an anhydrous or aqueous composition or in the form of an emulsion.

Preferably, the third composition is an anhydrous composition, in which case it advantageously comprises at least one lipophilic gelling agent, in particular at least one wax or pasty fat.

Preferably, the third composition is an aqueous composition, in which case it advantageously comprises at least one hydrophilic gelling agent.

Said embodiment is advantageous in that it provides protection for the product of the invention, even improves the preserving thereof and maintains the freshness thereof up until the film is removed by the user.

### Kit

In one particular embodiment, a product of the invention is an extemporaneous product i.e. it is the user, at the time of first use of the product, who will perform contacting between the first composition and the second composition.

Therefore, the invention also concerns a kit comprising:
at least one first composition such as previously described;
at least one second composition such as previously described;
optionally, at least one third composition such as previously described; and
optionally, at least one container.

In the remainder of the present description, and having regard to the field under consideration, a container can indifferently be designated by «container», «packaging» or «primary packaging».

In one particular embodiment, the first composition may have high contents of biological/cosmetic active substances such as described below. Therefore the first composition can be termed a «booster».

In one particular embodiment, the first composition in particular if it has a configuration of honeycomb type as previously described, may comprise a plurality of sections in particular polygonal sections, each of the sections defining a fractionable perimeter allowing the separation of at least one section from the remainder of the first composition. Therefore, in this embodiment, each section of the first composition can correspond to a single dose. This embodiment is advantageous in that it provides the user with the possibility of using a fresh product each time of use.

The invention further concerns the use of the above-described kit to obtain a product of the invention.

The invention further concerns a method for preparing a product of the invention comprising at least the steps of:
providing a kit such as previously described;
(2) providing a container; and
(3) in the container, placing in contact the first composition and second composition, even covering the first and second compositions with at least one third composition, after which the product of the invention is obtained.

Optionally, the step to cover the first and second compositions with a third composition may require prior heating of the third composition to a temperature able to impart thereto a temporary liquid nature.

In a first variant of embodiment, the first composition is already present in the container, in which case the user pours the second composition, then optionally the third composition, into the container to obtain the product of the invention.

Advantageously, the second composition-before being mixed with the first composition—is packaged in another adapted container, adapted in particular to the liquid nature of the second composition, and in particular chosen from among a tube, Berlingo, Doypack, dropper.

In a second variant of embodiment, the second composition is already present in the container, in which case the user places the first composition then optionally the third composition in the container to obtain the product of the invention.

The solid nature of a first composition, before being mixed with the second composition, advantageously allows obviating the need for conventional containers. Therefore the first composition can be packaged in simple wrapping such as a sheet of paper.

In one particular variant of embodiment, the formulation of the first composition and/or of the second composition is adjusted to ensure reducing of the solidity of the first composition in the presence of the second composition, preferably over a period of time of less than 6 hours, in particular less than 3 hours, more particularly less than 1 hour and better still of less than 30 minutes. This variant is advantageous in that gives access to products having further improved comfort and sensory perception when dispensing and on application.

In this variant, the first composition advantageously comprises at least one disintegrating agent, in particular such as described in the patent application filed under N° FR2212636.

Said adjustments of formulations are within the general reach of skilled persons.

In one particular embodiment, the kit may comprise a container in which the first and second compositions, even also the third composition, are packaged in separate compartments.

Preferably, a kit of the invention may also comprise at least one means of application/administering.

If the product is intended for cosmetic use, a kit of the invention may also comprise at least one means for application onto keratin material, chosen for example from the group formed by spatulas, different brushes, puffs or sponges.

In one particular variant, the kit may comprise several first compositions, the same or different and/or several second compositions the same or different, and optionally several third compositions the same or different, and/or at least one application means.

Therefore, the invention also concerns a kit comprising several first compositions in the form of single doses comprising different cosmetic/biological active substances and/or different contents of cosmetic/biological active substances.

Said kit of the invention therefore advantageously enables easy adaptation by a user according to desired effect.

For a cosmetic product, this particular embodiment enables the user to decide on the desired cosmetic effect, in particular as a function of state of mind (mood), dress style and/or time of day.

### Fatty Phase

Advantageously, the fatty phase of a first and/or third composition, in particular the solid phase of the first composition, advantageously has a melting point of between 50° C. and 100° C., preferably between 60° C. and 90° C.

The melting point can be measured by differential scanning calorimetry (DSC), for example with the calorimeter sold under the trade name “DSC Q2000” by TA Instruments. The protocols for preparing samples and measurement are the following: a 5 mg sample to be tested, previously heated to 80° C. and sampled under magnetic stirring using a spatula that is also heated, is placed in a hermetically sealed aluminium capsule or sample pan. Two tests are conducted to ensure reproducibility of results. Measurements are taken on the calorimeter mentioned above. The furnace is flushed with nitrogen. Cooling is ensured with an RCS 90 heat exchanger. The sample is brought to 20° C. and subjected to the following protocol: a first temperature rise from 20° C. to 130° C. at a heating rate of 5° C./minute, cooling from 130° C. to −80° C. at a cooling rate of 5° C./minute, and finally a second temperature rise from −80° C. to 130° C. at a heating rate of 5° C./minute. During the second temperature rise, the variation is measured in the difference between the power absorbed by the empty sample pan and by the sample pan containing the sample, as a function of temperature. The melting point of the compound is the value of the temperature corresponding to the peak of the curve representing the variation in difference of absorbed power as a function of temperature. The end melt temperature corresponds to the temperature at which 95% of the sample has melted.

A fatty phase comprises at least one lipophilic gelling agent.

A first and/or second and/or third composition may comprise at least one lipophilic gelling agent, preferably heat-sensitive, and optionally at least one oil.

### Lipophilic Gelling Agent

A lipophilic gelling agent can be chosen from among organic or mineral, polymeric or molecular lipophilic gelling agents; fats solid at ambient temperature and pressure; and mixtures thereof.

Fats solid at ambient temperature and pressure are preferably chosen from among at least one wax, at least one pasty fat, at least one butter, and mixtures thereof.

Advantageously, a lipophilic gelling agent is a heat-sensitive gelling agent, namely one which reacts to heat, and in particular a gelling agent solid at ambient temperature and liquid at a temperature higher than 50° C., preferably higher than 60° C., better still higher than 70° C. Preferably, a heat-sensitive lipophilic gelling agent of the invention has a melting point of between 50° C. and 130° C., more preferably between 60° C. and 120° C.

As lipophilic gelling agent(s), mention can be made for example of those described in WO2021234135.

In one particularly preferred embodiment, the lipophilic gelling agent is chosen from among Castor Oil/IPDI Copolymer (and) Caprylic/Capric Triglyceride marketed in particular under the tradename Estogel M by PolymerExpert, Hydrogenated Castor Oil/Sebacic Acid Copolymer and the derivatives thereof particularly those marketed under the tradenames Estogel Green (or Estogel G) and Estogel Green 40 by PolymerExpert, Caprylic/Capric Triglyceride (and) Polyurethane-79 in particular the one marketed under the tradename OILKEMIA™ 5S polymer by Lubrizol, Trihydroxystearin in particular as marketed under the tradename THIXCIN@ R by Elementis Specialties, and mixtures thereof, and more preferably it is Castor Oil/IPDI Copolymer (and) Caprylic/Capric Triglyceride.

In particular, a first and/or second and/or third composition may comprise from 10% to 90%, preferably from 20% to 80%, in particular from 30% to 70%, and better still from 40% to 60% by weight of lipophilic gelling agent(s) relative to the total weight of the composition comprising the same.

Persons skilled in the art are able to adjust the nature and/or content of lipophilic gelling agent(s), in particular to pay heed to the solid or liquid nature of the composition under consideration.

### Oils

A first and/or second and/or third composition may further comprise at least one oil. By «oil» it is meant a fat that is liquid at ambient temperature and atmospheric pressure.

As oils of the invention, mention can be made for example of:
hydrocarbon oils of vegetable origin, such as described below;
hydrocarbon oils of animal origin such as perhydrosqualene and squalane;
synthetic esters and ethers, in particular of fatty acids, such as oils of formulas R¹COOR² and R¹OR² where R¹ represents the remainder of a C₈ to C₂₉ fatty acid, and R² represents a C₃ to C₃₀ branched or non-branched hydrocarbon chain such as Purcellin oil, isononyl isononanoate, isodecyl neopentanoate, isostearyl neopentanoate, isopropyl myristate, octyldodecyl myristate, ethyl-2-hexyl palmitate, octyl-2-dodecyl stearate, octyl-2-dodecyl erucate, isostearyl isostearate; hydroxylated esters such as isostearyl lactate, octylhydroxystearate, octyldodecyl hydroxystearate, diisostearyl-malate, triisocetyl citrate, heptanoates, octanoates, fatty alcohol decanoates; polyol esters such as propylene glycol dioctanoate, neopentylglycol diheptanoate and diethyleneglycol diisononanoate; and the esters of pentaerythritol such as pentaerythrityl tetrabehenate (DUB PTB) or pentaerythrityl tetraisostearate (Prisorine 3631);
linear or branched hydrocarbons or mineral or synthetic origin, such as paraffin oils whether or not volatile and derivatives thereof, Vaseline, polydecenes, hydrogenated polyisobutene such as Parleam oil;
silicone oils, such as polydimethylsiloxanes (PDMS) for example whether or not volatile, having a linear or cyclic silicone chain, liquid or pasty at ambient temperature, in particular cyclopolydimethylsiloxanes (cyclomethicones) such as cyclohexasiloxane and cyclopentasiloxane; polydimethylsiloxanes (or dimethicones) comprising alkyl, alkoxy or phenyl groups either pendant or at the end of the silicone chain, groups having 2 to 24 carbon atoms; phenyl silicones such as phenyltrimethicones, phenyldimethicones, phenyltrimethylsiloxydiphenyl-siloxanes, diphenyl-dimethicones, diphenylmethyldiphenyl trisiloxanes, 2-phenylethyltrimethyl-siloxysilicates, and polymethylphenylsiloxanes;
fatty alcohols having 8 to 26 carbon atoms such as cetyl alcohol, stearyl alcohol and mixture thereof (cetylstearyl alcohol), or octyldodecanol;
partially hydrocarbon-based and/or silicone-based fluorinated oils such as those described in document JP-A-2-295912;
and mixtures thereof.

As oils, mention can be made for example of those described in WO2021234135.

In particular, a first and/or second and/or third composition may comprise from 10% to 97%, preferably from 20% to 90%, in particular from 30% to 80%, better still from 40% to 70% by weight of oil(s) relative to the total weight of the composition comprising the same.

### Aqueous Phase

A first and/or second and/or third composition may comprise at least one aqueous phase.

An aqueous phase at least comprises water. In addition to distilled or deionized water, a water suitable for the invention can also be a natural spring water or floral water.

In one embodiment, the mass percentage of water in the aqueous phase is at least 30%, preferably at least 40%, in particular at least 50%, and better still at least 60%, in particular between 70% and 98%, and preferably between 75% and 95%, relative to the total mass of the aqueous phase.

The aqueous phase may also comprise at least one base. It may comprise a single base or a mixture of several different bases The presence of at least one base in said aqueous phase can contribute in particular to increasing the viscosity thereof, in particular when this aqueous phase also comprises at least one pH-dependent hydrophilic gelling agent, for example a carbomer.

In one embodiment, the base included in the aqueous phase is a mineral base. In one embodiment, the mineral base is chosen from the group formed by hydroxides of alkali metals and hydroxides of alkaline-earth metals. Preferably, the mineral base is an alkali metal hydroxide and in particular NaOH. In one embodiment, the base included in the aqueous phase is an organic base. Among organic bases, mention can be made for example of ammonia, pyridine, triethanolamine, aminomethylpropanol, or triethylamine.

An aqueous phase may comprise from 0.01% to 10% by weight, preferably from 0.01% to 5% by weight, more preferably from 0.02% to 1% by weight of base, preferably of mineral base and in particular NaOH, relative to the total weight of the aqueous phase.

An aqueous phase is preferably in gel form. An aqueous phase preferably has viscosity of between 400 mPa·s and 100000 mPa·s, preferably between 800 mPa·s and 30000 mPa·s, such as measured at 25° C. according to the above-described method.

### Additional Compounds

A product of the invention, in particular the first and/or second and/or third composition(s) may further comprise at least one additional compound differing from the above-mentioned lipophilic gelling agents and oils.

A product of the invention, in particular the first and/or second and/or third composition(s) may further comprise powders; glitter; colouring agents chosen in particular from among colouring agents whether or not water-soluble, whether or not liposoluble, organic or inorganic; materials with optical effect, liquid crystals, and mixtures thereof; particulate agents insoluble in the fatty phase; fragrances; preserving agents; humectants; fillers in particular fillers with blur effect; UV filters; effervescent agents; diluents; disintegration promoters; disintegrants (e.g. croscarmellose, carboxymethylcellulose, . . . ); stabilizers; chelating agents; emollients; modifying agents chosen from among texturizing agents (or gelling agents) and in particular hydrophilic gelling agents; modifiers of pH, osmotic force, and/or refractive index modifiers etc. . . . or any usual cosmetic additive; and mixtures thereof.

By «filler», in the meaning of the invention, it is meant solid, colourless or white particles of any shape which are in insoluble form and dispersed in the medium of the composition. Of mineral or organic type, they allow the imparting of body or rigidity and/or softness and uniformity of deposit, in particular with regard to makeup, and improved stability with regard to exudation and non-migratory properties after application, and/or matt finish and/or coverage.

By «particulate agents insoluble in the fatty phase» in the meaning of the invention it is meant the group formed by pigments, ceramics, polymers in particular acrylic polymers, and mixtures thereof.

As hydrophilic gelling agents, mention can be made for example of those described in FR3041251.

A product of the invention, in particular the first and/or second and/or third composition(s), may further comprise at least one biological/cosmetic active substance. As biological/cosmetic active substance of the invention, mention can be made of hydrating agents, vitamins, healing agents, antiwrinkle or anti-age agents, depigmenting agents, peeling agents, antioxidants, active substances stimulating synthesis of dermal or epidermal macromolecules, skin relaxants, anti-perspirants, soothing agents and/or anti-age agents, and mixtures thereof.

A first composition may further comprise at least one solvent able to evaporate fully or partially during the production process as described previously. As example of solvent, an apolar or scarcely polar organic solvent can be cited e.g. cyclohexane, THF (tetrahydrofuran), and mixtures thereof.

Evidently, those skilled in the art will take care to choose the raw materials, ingredient(s), active substance(s) and/or additional compound(s) described above and/or the quantity thereof so that the advantageous properties of the product of the invention are not or are not substantially altered by the envisaged addition. Also, skilled persons will ensure that the chosen type and/or quantity of raw materials, ingredient(s), active substance(s) and additional compound(s) described above are chosen as a function of the aqueous or fatty nature of the composition, even of the phase under consideration and/or having regard to the production method thereof. These adjustments lie within the general knowledge of persons skilled in the art.

### Use

A product of the invention can be a cosmetic, pharmaceutical, nutraceutical or food product.

A product of the invention can be a topical or oral product, even orodispersible, preferably topical. Preferably a product of the invention is not an oral or orodispersible product.

Preferably, a product of the invention is a cosmetic product, and preferably dedicated to the care and/or makeup of a keratin material in particular the skin and/or lips.

In particular, a product of the invention can be a cream for example, a lotion, serum and gel for the skin (hands, face, feet, etc.), a hair care product (hair dyes and bleaches), a hair conditioning product (lotions, creams, oils), a hair styling product (lotions, lacquers, brilliantine), a shaving product (soaps, foams, lotions, etc.), a product intended to be applied to the lips, a skin whitening product, an antiwrinkle product. In particular, the cosmetic compositions of the invention can be an anti-age serum, youth serum or hydrating serum.

A product of the invention can be a BB Cream or CC cream and therefore additionally comprise at least one pigment.

Finally, the present invention also concerns a therapeutic method comprising at least one step to apply or administer a least one product of the invention.

Finally the present invention also concerns a non-therapeutic cosmetic treatment method, in particular for the care and/or makeup of a keratin material in particular the skin and/or lips, comprising at least one step to apply to said keratin material at least one product such as previously described.

In the entire description, the expression «comprising one» is to be construed as being synonymous with «comprising at least one», unless otherwise specified. The expressions «between . . . and . . .», «from . . . to . . .» and «ranging from . . . to . . .» are to be understood as including the limits, unless otherwise specified.

### EXAMPLES

### Example 1-Preparation of the First Solid Composition

TABLE 1

| NAME | INCI | % | Phase |
|---|---|---|---|
| LABRAFAC CC | Caprylic/CapricTriglyceride | q.s.* | A |
| CASTOR OIL | RicinusCommunis(Castor) | 8.00 | A |
| | SeedOil | | |
| ESTOGEL M | Castor Oil/IPDI Copolymer | 30.00 | A |
| | (and) Caprylic/ | | |
| | CapricTriglyceride | | |
| ESTOGEL GREEN | HydrogenatedCastor Oil/ | 8.00 | A |
| | SebacicAcid Copolymer | | |
| LUSPLAN DD-DA7 | Dimer DilinoleylDimer | 3.70 | A |
| | Dilinoleate | | |
| Coenzyme Q10 | Ubiquinone | 0.2 | B |
| COPHEROL 1250 C | Tocopheryl Acetate | 0.50 | B |
| FRAGRANCE | Fragrance | 0.50 | B |

| | |
|---|---|
| Total | 100 |

In sufficient amount

### Protocol for Preparing the First Solid Composition:

Place the ingredients of phase A in a container and heat the mixture under agitation to 100° C. until homogenization.

Cool to 80° C. and add the ingredients of phase B under agitation until homogenization.

### Extrusion of the First Solid Composition:

The mixture in Table 1 is injected into an extruder provided in a first case with a die such as illustrated in FIG. 4 in the patent application filed under n° FR2203882, and in a second case a comparative die such as illustrated in FIG. 5 of the patent application filed under n° FR2203882;

On leaving the die, the respectively extruded bars are cut to obtain the first solid compositions described in Table 2 below.

Two first compositions are obtained, respectively in the form of a solid composition having a honeycomb structure (test A) and a solid composition having a general shape identical to that of Test 1 but devoid of cavity (test B).

TABLE 2

| | Test A | Test B |
|---|---|---|
| | Solid composition | Bulk solid composition |
| | with cavities | (i.e. without cavity) |
| Architecture | Invention | Comparative |
| Weight (in g) | 6 | 6 |
| Volume (in cm³) | 25 | 6.25 |
| Porosity (%) | >70 | 0 |

### Example 2-Preparation of the Second Liquid Composition

TABLE 3

| Fluid | Name | INCI | % w/w |
|---|---|---|---|
| OF | Osmotic water | — | q.s.* |
| | Glycerol | Glycerine | 17.31 |
| | Zemea | Propanediol | 5.77 |
| | Butylene Glycol | Butylene glycol | 5.77 |
| | Microcare PTG | Pentyleneglycol | 2.3 |
| | Microcare PE | Phenoxyethanol | 0.92 |
| | Carbomer Tego 340 FD | Carbomer | 0.25 |
| | EDETA | EDTA disodium | 0.04 |
| | NaOH | Sodium hydroxide | 0.01 |
| | Total | | 100 |
| Base | Osmotic water | Water | q.s .* |
| | NaOH | Sodium hydroxide | 0.5 |
| | Total | | 100 |
| IF | Lanol 99 | Isononyl | q.s .* |
| | | Isononanoate | |
| | Rheopearl KL2 | Dextrin Palmitate | 10 |
| | KF 8004 | Amodimethicone | 0.5 |
| | Phat Black DC 9206 | CI 61565, | 0.15 |
| | | CI 60725, CI 26100 | |

| | |
|---|---|
| Total | 100 |

In sufficient amount

### Protocol for Preparing the Second Liquid Composition

The preparation of the fluids is described in Example 3 of patent application WO2017046305. The second composition is produced by non-microfluidic process via use of a Rayneri mixer.

Alternatively, the second composition can also be produced by microfluidic process, for example such as described in WO2015055748, in which case the second composition is an emulsion in which the dispersed drops of the fatty phase are macroscopic and monodisperse.

### Example 3-Comparative Study

A 30 ml container is half filled with the second composition in Example 2.

The first composition of Example 1 (Test A or B) is then added, after which a comparative product is obtained and a product of the invention similar to the one illustrated in FIG. 1.

### Application:

This step was performed on a panel of 30 women aged between 22 and 47 years (hereafter the «candidates»), 5 groups being formed with 6 women in each group.

In each group, the candidates dispensed the product with a spatula and applied the dispensed product to the skin of the face.

Each of the groups used the five products described in Table 5 below, but each time in a different order, and product performance was evaluated in terms of (i) ease of dispensing, (ii) homogeneity of deposit, (iii) comfort on application, and (iv) ease of complying with the prescribed routine of use (i.e. one application in the morning and evening for 20 days).

Each of the groups also gave their opinion on product design and galenic form.

### Score Criteria:

TABLE 4

| SCORE CRITERIA | 0 | 1 | 2 |
|---|---|---|---|
| Ease of dispensing | Satisfactory | Moderate | Poor |
| Homogeneity of | | | |
| deposit | | | |
| Comfort on | | | |
| application | | | |
| Ease of compliance | | | |
| with prescribed | | | |
| routine of use | | | |
| Product design | | | |
| Galenic form | | | |

The averages of the results obtained with the five groups are given in following Table 5, it being pointed out that similar trends are observed between the five groups.

TABLE 5

| | | | | | Test 5 |
|---|---|---|---|---|---|
| | Test 1 | Test 2 | Test 3 | Test 4 | Inven- |
| | Compar. | Compar. | Compar. | Compar. | tion |
| First | Test B | — | Test B | Test A | Test A |
| composition | | | | | |
| Second | — | Table 3 | Table 3 | — | Table 3 |
| composition | | | | | |
| Ease of | 2 | 0 | 2 | 1 | 0 |
| dispensing | | | | | |
| Homogeneity | 1 | 0 | 1 | 1 | 0 |
| of deposit | | | | | |
| Comfort on | 2 | 0 | 1 | 1 | 0 |
| application | | | | | |
| Intent to | 2 | 1 | 1 | 1 | 0 |
| comply with | | | | | |
| prescribed | | | | | |
| routine of use | | | | | |
| Design | 1 | 1 | 1 | 1 | 0 |
| Galenic form | 2 | 1 | 1 | 1 | 0 |

With comparative test 1, it is difficult to dispense a sufficient amount of composition with the spatula, and therefore to evaluate homogeneity of deposit and comfort on application.

With comparative test 2, it is observed that handling of the product is «slippery» under the fingers.

Comparative test 3 is not satisfactory insofar as dispensing of the first composition with the spatula remains difficult. In addition, the block slips and is difficult to break with the spatula in the middle of the cream.

With comparative test 4, the results obtained lie within the average.

Test 5 is the test which exhibits the best results. The presence of cavities in the first composition gives access to a comfortable product with innovative galenic form and design. Sensory perception on dispensing is judged to be unique and innovative, translating as alternation between softness and glide with the second composition and resistance with the first composition (=saccade-like dispensing).

Test 5 is the test which most appealed to the panel and is therefore likely to ensure the strictest compliance in terms of use, and hence the best results in terms of desired effects.

1. A product comprising at least one first composition and at least one second composition, wherein:
the first composition is a solid composition comprising at least one cavity such that the first composition has porosity greater than or equal to 20% by volume relative to the total volume of the first composition,
the second composition is a liquid composition,
and wherein all or part of the second composition is housed in all or part of the cavities of the first composition.

2. The product according to claim 1, wherein the second composition has viscosity higher than or equal to 5000 cps.

3. The product according to claim 1 wherein the first composition has porosity greater than or equal to 30% by volume, relative to the total volume of the first composition.

4. The product according to claim 1 wherein the cavity/cavities of the first composition are through cavities.

5. The product according to claim 1 wherein the first composition comprises one or more cavities, which is/are the same or different.

6. The product according to claim 1 wherein the cavity/cavities of the first composition are of round, oblong, ovoid, triangular, polygonal, honeycomb shape, or in tessellated form (or partitioned).

7. The product according to claim 1 wherein the ratio between the volume of the cavity/cavities and the total volume of the first composition is between 0.2 and 0.9.

8. The product according to claim 1 wherein the product further comprises at least one third composition dedicated to closing all or part of the cavities of the first composition.

9. The product according to claim 1 wherein the first and/or second composition(s) comprise at least one lipophilic gelling agent and optionally at least one oil.

10. The product according to claim 1 wherein the first and/or second composition(s) further comprise at least one aqueous phase.

11. The product according to claim 1 wherein the product has a weight ratio of «first composition(s)/second composition(s)» of between 0.01 and 10.

12. The product according to claim 1 wherein the product is a cosmetic, pharmaceutical, nutraceutical or food product.

13. The product according to claim 1 wherein the product is a cosmetic product dedicated to the care and/or makeup of a keratin material.

14. The product according to claim 1 wherein the product is a topical or oral product.

15. A kit comprising:
at least one first composition and at least one second composition according to claim 1;
optionally at least one third composition dedicated to closing all or part of the cavities of the first composition; and
optionally, at least one container.

16. The kit according to claim 15, wherein the first composition, if it has a configuration of honeycomb type, comprises a plurality of sections, each of the sections defining a fractionable perimeter allowing the separation of at least one section from the remainder of the first composition.

17. The kit according to claim 15, wherein the kit further comprises application/administering means.

18. The kit according to claim 15, wherein the kit further comprises at least one application means onto keratin material chosen from the group formed by spatulas, various brushes, puffs or sponges.

19. A method for preparing the product according to claim 1, comprising at least the steps of:
a. providing a kit comprising:
the at least one first composition
the at least one second composition,
optionally at least one third composition dedicated to closing all or part of the cavities of the first composition, and
optionally, at least one container;
b. providing a container; and
c. in the container placing in contact the first composition and the second composition, even covering the first and second compositions with at least one third composition, after which a product of the invention is obtained.

20. A non-therapeutic cosmetic treatment method for the care of a keratin material, comprising at least one step to apply to said keratin material at least one product according to claim 1.
